(19) United States
(12) Patent Application Publication
(10) Pub. No.: US 2004/0096831 A1
(43) Pub. Date: May 20, 2004

(54) Method and system for hybridizing biological materials

(75) Inventor: Owen P.L. Hughes, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Hybridization Systems, Inc. (a California corporation), Davis, CA

(21) Appl. No.: 10/298,232

(22) Filed: Nov. 15, 2002

(51) Int. Cl. C12Q 1/68; C12M 1/34

(52) U.S. Cl. 435/6; 435/287.2

(57) ABSTRACT

A method and apparatus for facilitating molecular associations. The method involves use of independently controlled temperature zones; simultaneously promoting optimized reaction and optimized maintenance of reaction components, while also providing for convection driven mixing.

### CROSS-REFERENCES TO RELATED APPLICATIONS

### STATEMENT AS TO RIGHTS TO INVENTIONS MADE UNDER FEDERALLY SPONSORED RESEARCH OR DEVELOPMENT

### REFERENCE TO A “SEQUENCE LISTING,” A TABLE, OR A COMPUTER

### PROGRAM LISTING APPENDIX SUBMITTED ON A COMPACT DISK.

### BACKGROUND OF THE INVENTION

This invention relates generally to processing chemicals/materials, including biological materials. More particularly, the invention provides methods and systems for which isothermal reaction conditions and/or thermal conditions are not optimal or are desirably optimized. More particularly, the invention provides technique including methods and systems for conducting chemical or biochemical reactions involving molecular association. Examples of molecular association reactions include hybridization assays in which the self-association properties of nucleic acids are used to identify or quantify genes or gene products in a given sample. Other examples of molecular association reactions include enzyme/substrate assays, catalyst/substrate reactions, reactive chemical reactions and antibody/antigen reactions. The present invention has utility in fields relating to biology, chemistry, biochemistry, and others.

Tests using DNA (deoxyribonucleic acid) hybridization have become common in analytics and medical diagnostics. Nucleic acids can be used as specific detection agents for organisms and for the diagnosis of diseases. A basic property of DNA-self-association or DNA hybridization between the two complementary strands that include the DNA double helix—is exploited in many of these tests. DNA has two complementary strands. The two strands of DNA can be separated by heat and when cooled each strand will reassemble—hybridize—with its complementary partner to reform a double helix. In this manner specific genes can be tested by labeling one strand with something that can be detected, then letting hybridization determine if the complementary target gene is present.

DNA hybridization was used to estimate genome size and complexity, and later utilized for identification and quantification of gene activity in a process named after its inventor—Ed Southern. Southern blots involve transferring DNA fragments to a membrane—then probing the membrane with detectable DNA strands of a specific gene of interest. Simplified and expanded, Southern blots have become DNA Chips or Microarrays which are finding wide application.

DNA microarrays are used to analyze expression of thousands of genes simultaneously. The technique includes immobilizing DNA from large numbers of genes on a solid substrate, such as a glass microscope slide, silicon chip, or plastic membrane. The DNA samples appear as an array of spots on the substrate. One can determine the origin of a particular DNA sample by knowing its position in the array. The technique typically uses self-association of the cDNA (copy or complement DNA) probes with their complements arrayed on a solid substrate to detect and quantify specific nucleotide sequences in the samples. cDNA is generated from the RNA (ribonucleic acid) message products expressed by active genes.

Under proper conditions, the cDNA probes hybridize or bind to their immobilized complement in the DNA microarray, resulting in hybrid DNA/cDNA strands. For each immobilized gene one can measure gene activity by measuring the intensity the amount of cDNA bound to each spot in the microarray by hybridization. Some of the technical limitations of DNA microarrays stem from limitations of the DNA hybridization process. DNA hybridization on DNA microarrays is slow and inefficient. Typically, hybridization requires 12-24 hours, and yields less than 35% hybridization efficiency of a specific probe to its target. The remaining 65% of the probe is stuck to itself, other probes within the sample mix, or the wrong location on the DNA microarray.

The basic biochemical reaction or process underlying Southern Blots—DNA hybridization—also underlies DNA chips or DNA Microarrays. Because of the shared biochemical reaction mechanisms, development of reaction conditions for microarrays based on experience from Southern blots has been fairly successful. The differences in implementation of a Southern blot and Microarray, however, have left a number of limitations on the performance of the DNA microarray hybridization reaction. Certain differences between microarray assays and Southern Blots include: 1) Southern blots are generally “probed” with a single labeled gene, while DNA microarrays are generally probed with the entire expressed compliment of genes (thousands of probes for a DNA microarray as opposed to one on a Southern Blot). 2) DNA microarray assays generally have limiting amounts of the labeled probe while Southern blots are probed with an excess of the gene of interest. 3) DNA microarray assays are generally performed in substantially reduced reaction volumes.

Microarray hybridization has been performed at single temperature conditions that is a compromise between a temperature low enough to allow for proper hybridization (signal), yet high enough to reduce incorrect hybridization (noise). This compromise leads to limitations on hybridization efficiency. Incorrect hybridization of the test material is a factor limiting hybridization—often requiring higher concentrations of the test material and limiting overall sensitivity of the test. These incorrect hybridization interactions include: A.) Hybridization of the test molecules to themselves. B.) Hybridization between different molecules in test solution. C.) Hybridization to the incorrect spot/gene on the array. D.) Non-specific sticking to the array, increasing general background noise. Numerous limitations exist with these conventional DNA hybridization processes.

A number of attempts have been made to improve the conventional DNA hybridization processes. The small reaction volumes used with DNA microarrays results in poor mixing of the reaction-which both increases sample requirements and increases inconsistency of the results. Various methods used to increase mixing are outlined in U.S. Pat. No. 5,856,174, which generally utilizes a moving bubble in the reaction chamber or sonic agitation to promote mixing. Another method for increasing mixing can be seen in U.S. Pat. No. 6,238,910, which generally utilizes opening and closing of a valve for agitation of the reaction mix.

Small reaction volumes are used in microarray hybridization in an attempt to increase the effective concentration of the sample and thereby increase the efficiency of hybridization. With microarray hybridization generally done under probe-limiting conditions the need to use small reaction volumes exacerbates the need for reaction mixing. Another approach to improving the hybridization process is to modify the reaction solution with chemical agents intended to facilitate hybridization of complementary strands, and aid the denaturation/melting of inappropriate interactions. Commonly used chemical agents include detergents, Denhart's Solution, and formamide, and salts. An example of this approach is outlined in U.S. Pat. No. 6,045,996—“Hybridization assays on oligo nucleotide arrays”. Although such approach was fairly well explored with Southern blots, it has not resulted in significant new performance gains for DNA microarrays.

Another approach for improving DNA hybridization is to physically change the properties of the hybridizing elements. Such approach links the target DNA to the solid substrate via a flexible and specific covalent chemical linkage with one end of the target DNA molecule. It is desired to decrease the steric hindrances to hybridization that may be encountered if the target DNA is attached the solid substrate via non-specific interactions along the length of the target molecule. This approach has had limited success in improving hybridization. Yet another approach involves modification of the target molecules such that hybridization the kinetics is improved. Such modified approach reports substantial improvement in hybridization, but often requires users to modify their microarray gene library for every gene being tested (“Rapid renaturation of complementary DNA strands mediated by cationic detergents: A role for high-probability binding domains in enhancing the kinetics of molecular assembly processes”, Proc. Natl. Acad. Sci. USA, 88:8237-8241, September 1991). Such approach is expensive and inflexible.

From the above, it is seen that techniques for controlling process parameters of small reaction vessels are highly desirable—and methods and systems for improving molecular association based reaction would have utility.

### BRIEF SUMMARY OF THE INVENTION

According to the present invention, techniques including methods and systems for processing materials are provided. Further, this invention provides for a means of controlling local concentration of reactants. More particularly, the invention provides technique including methods and systems for processing reactions using molecular association. Merely by way of example, the invention is applied to molecular association for diagnostics using biological arrays of materials on arrays of spots on substrates. But it would be recognized that the invention could also be applied to other molecular associative reactions such as enzyme/substrate assays, catalyst/substrate reactions, reactive chemical reactions and antibody/antigen reactions.

In embodiment, the invention provides for means of controlling local temperature and reactant concentration at more than one location in a reaction chamber. Zone based control of temperature allows for optimal reaction conditions in situations where a single reaction temperature is sub-optimal. Further, zone based temperature control allows for temperature difference driven mixing or the reaction. Additionally, zne based, volume independent concentration control enables manipulation of concentration conditions for optimization of reaction conditions.

In a specific embodiment, the invention includes a system for processing biological materials for reactions using molecular association. The system includes a first substrate comprising a surface. The surface includes an array of spots. Each of the spots comprises biological material coupled to the substrate. A second substrate is coupled to the first substrate. The second substrate is separated from the first substrate by a predetermined distance. The system also has a fluid disposed between the first substrate and the second substrate and occupying the predetermined distance. A first energy source is coupled to the first substrate to provide a first desired temperature to the first substrate. A second energy source is coupled to the second substrate to provide a second desired temperature to the second substrate.

In an alternative specific embodiment, the invention includes a method for processing biological materials for diagnostics using molecular association. The method includes disposing fluid between a first substrate and a second substrate. The first substrate is coupled to the second substrate. The first substrate comprises a surface. The surface includes an array of spots. Each of the spots has a biological material coupled to the substrate. The second substrate is separated from the first substrate by a predetermined distance, whereupon the fluid occupies the predetermined distance and overlies each of the spots in the array. The method also includes applying a first energy to the first substrate to cause a first desired temperature to the first substrate and applying a second energy to the second substrate to cause a second desired temperature to the second substrate. The method causing a temperature gradient between the first substrate and the second substrate through the fluid occupied within the predetermined distance, whereupon the first temperature enhancing a first reaction process at the array of spots and the second temperature enhancing a second reaction process at the second substrate.

In an alternative specific embodiment, the invention includes a method for processing biological materials for diagnostics using molecular association. The method includes disposing fluid between a first substrate and a second substrate. The first substrate is coupled to the second substrate. The first substrate comprises a surface, which has an array of spots. Each of the spots comprises biological material coupled to the substrate. The second substrate is separated from the first substrate by a predetermined distance, whereupon the fluid occupies the predetermined distance and overlies each of the spots in the array. The method applies a first energy to the first substrate to cause a first desired temperature to the first substrate and applies a second energy to the second substrate to cause a second desired temperature to the second substrate. The method causes a temperature gradient between the first substrate and the second substrate through the fluid occupied within the predetermined distance, whereupon the first temperature enhancing a first reaction process at the array of spots and the second temperature enhancing a second reaction process at the second substrate.

In a specific embodiment, the invention includes a system for processing reactants by manipulating the local concentration of reactants within a reaction chamber in a volume independent manner. More specifically, and external force is applied to the reactants to move them into the desired reaction zones and thereby increase or decrease their local concentration in a volume independent manner. In one embodiment of this invention the motive force on reactant is based on electronic charge, with reactants being repelled by like charge and attracted to opposite charge. In another embodiment of this invention the means of applying external force is mass based, with gravity or centrifugation used to drive reactants to the desired zone. In another embodiment of this invention the means of applying external force it is magnetically based, with magnetic fields used to drive reactants to the desired zone.

The system includes a first substrate comprising a surface. The surface includes an array of spots. Each of the spots comprises biological material coupled to the substrate. A second substrate is coupled to the first substrate. The second substrate is separated from the first substrate by a predetermined distance. The system also has a fluid disposed between the first substrate and the second substrate and occupying the predetermined distance. A first energy source is coupled to the first substrate to provide a first desired temperature to the first substrate. A second energy source is coupled to the second substrate to provide a second desired temperature to the second substrate.

This invention pertains to molecular associations that occur at or near a localized association surface. The essence of one aspect of this invention lays in use of different temperatures at the association surface the reaction mixture verses the opposing reaction chamber surface. The difference in temperature can be used to promote mixing within the reaction chamber (via thermal convective currents). Additionally, by having different temperatures in the reaction chamber versus the reaction surface, the un-reacted components can be maintained at better suited temperatures for the un-reacted components, yet allow for an optimal temperature for molecular association that the reaction surface. In addition to the control of reaction chamber zone temperature, this invention also provides a means to improve reaction conditions by manipulating local concentration of reactants in a volume independent fashion. Manipulation of local concentration enables greater efficiency/yield for the reaction while improving the users ability to reduce background and waste. Examples of various embodiments are provided more fully below:

DNA Hybridization Engine Embodiment:

Most specifically, this invention entails the use of independently controlled temperature at the opposing faces of a hybridization chamber to provide for easy temperature control and a temperature differential between the hybridization surface and the general reaction chamber. This temperature differential is used to simultaneously provide optimal hybridization temperature at the hybridization surface while also providing for a higher temperature capable of melting undesired secondary structure. Further, this invention can entail the use of external forces (i.e. electronic, magnetic, or mass dependant) to control the local concentration of reactants.

One embodiment of this invention entails the use of independently controlled temperature at the opposing faces of a hybridization chamber to provide for easy temperature control and a temperature differential between the hybridization surface and the general reaction chamber.

In one application of this invention, the reaction surface holds nucleic acid molecules or their analogs to be reacted (hybridized) with their complementary nucleic acid molecules in the reaction mix. This is the situation of a DNA microarray being hybridized with labeled CDNA or mRNA in order to asses gene expression, gene presence, or gene sequence in cells of interest.

In a simple embodiment of this invention designed to facilitate DNA hybridization, two opposing temperature controlled plates hold opposing glass slides. These opposing microarray slides form a hybridization chamber by sandwiching an o-ring or gasket like seal.

The hybridization surface can be brought to a hybridization optimal temperature, while the opposing surface could be set at a higher temperature. This allows for hybridization to occur at the hybridization optimal temperature, and the nucleic acids not yet hybridized to their complement would be at a higher temperature. The higher temperature would facilitate the reaction in that it could melt inappropriate associations that reduce their ability to form the desired association with nucleic acids on the hybridization surface. Additionally, the higher temperature would facilitate the reaction in that it could promote mixing by thermal convection.

Further the hybridization surface could be situated vertically, to maximize thermal convective mixing, with the relatively cooler hybridization surface promoting a downward flow, and the hotter chamber backing promoting an upward flow.

The temperature of the two opposing surfaces could be alternated if both opposing hybridization surfaces are used or if temperature fluctuations would be useful (e.g. wash step for reducing background). By cycling the hybridization surface between the high (wash) and low (hybridization) temperatures we obtain benefits of both specificity and efficiency of hybridization.

Further, two opposing temperature controlled plates holding opposing glass slides are maintained at opposing electrical charges. This allows for the concentration of net negatively charged molecules such as DNA to be concentrated away from the negatively charged temperature control plate and towards the positively charged temperature control plate. Control of the electrical charges on the opposing temperature control plates allows for cycling the DNA molecules between reaction chamber zones.

The benefits of this embodiment to DNA array hybridization would be increased sensitivity though more complete hybridization of the nucleic acids in the reaction mix with their complements on the hybridization surface. More complete hybridization is a result of freeing molecules sequestered in inappropriate associations, and a result of mixing to allow all molecules in the mix to contact their hybridization targets. Additionally, the same effects (mixing and probe rescue) also enable completion of the hybridization reaction in a far shorter time (˜1 hr vs. 12-24 hrs).

In an alternative application of this instrument, two opposing hybridization surfaces are used to form a hybridization chamber. Using two hybridization surfaces would have advantages in a number of situations: when the optimal hybridization temperature is not know, when repeated hybridization at differing stringency's (temperatures) is desirable, when hybridization at the same stringency, but using slide alternating hybridization/wash temperature cycles is desired-giving two hybridization's using the same amount of material as commonly used in a single hybridization.

Molecular Associations Facilitated by Invention:

Nucleic Acid or Nucleic Acid Analogs Hybridization

DNA Arrays

In Situ Hybridization

The independent control of temperature allows for increased speed and sensitivity through promotion of mixing and melting of incorrect hybridizations.

Antibody/Antigen

Lower reaction chamber temperature allows for decreased loss of probe and promotion of convective mixing. If the temperature is “cycled” increased sensitivity is the result of thermal disruption of incorrect associations.

Receptor/ligand

Just as with Antibody/antigen associations, lower reaction chamber temperature allows for decreased loss of probe and promotion of convective mixing. If the temperature is “cycled” increased sensitivity is the result of thermal disruption of incorrect associations.

Enzyme/Substrate

Just as with Antibody/antigen associations, lower reaction chamber temperature allows for decreased loss of enzyme or substrate and promotion of convective mixing. For enzyme/substrate interactions, temperature cycling can promote completion of the desired reaction at the permissive temperature, while allowing for loading, unloading, and issomerization at the other temperatures.

Reactants

Just as with Enzyme/substrate associations, lower reaction chamber temperature allows for decreased loss of reactants and promotion of convective mixing. Additionally, temperature cycling can promote completion of the desired reaction at the permissive temperature, while allowing for loading, unloading, and issomerization at the other temperatures.

It is a goal of this invention to improve processes involving molecular associations in general, and DNA hybridization in particular through the use of non-homogeneous reaction temperatures. For hybridization reactions, this invention provides for a method to combine both the “melt” or “wash” and “hybridize” temperatures in the same hybridization chamber. For other types of reactions, this invention provides an efficient means of providing mixing in low volumes, and optimal use of at least two temperatures. For example in some enzyme/substrate reactions, this invention provides for a method to combine both the “associate” and “react” temperatures in the same reaction chamber.

In the case of DNA hybridization, this invention provides for an instrument and systems employing temperature control of the hybridization surface and the test solution—thermodynamically optimizing the hybridization process. The instrument detailed in this invention sets the hybridization surface at a lower “hybridization” temperature, and sets the opposite face of the hybridization chamber at a higher “melt” temperature. This produces a temperature gradient, increasing away from the hybridization surface.

### BRIEF DESCRIPTION OF THE DRAWINGS

FIG. 1 is an overall system diagram according to an embodiment of the present invention;

FIG. 2 is a simplified side-view diagram of a system according to an embodiment of the present invention;

FIG. 3 is a simplified side-view diagram of a system according to an alternative embodiment of the present invention with a single reaction surface (microarray) and a non-reactive opposing face;

FIG. 4 is a simplified side-view diagram of a system according to an alternative embodiment of the present invention with temperature regulation achieved through the use of cooling or heating fluids or through the use of electrical resistive heating rather than Peltier effect thermoelectric temperature controllers;

FIG. 5 illustrates the mixing effect achieved with a temperature differential between zones of the reaction chamber.

FIG. 6 illustrates the local concentration control achieved with a motive force on reactants (electric, magnetic or density driven). In this case an electric charge is applied to the temperature regulation plates driving reactants away from the plate with like charge and towards the plate with opposite charge.

FIG. 7 illustrates the local concentration control achieved with a motive force on reactants. In this alternative embodiment a magnetic field is generated such that paramagnetic reactants can be driven towards either, or charged reactants can be driven perpendicular to a fluctuating magnetic field. Direction of the motive force may be controlled by controlling the polarity of electricity flowing through the electromagnets. In yet another alternative embodiment, reactants may be driven to desired locations based on density using gravity or centrifugation.

FIG. 8 is a simplified flow diagram of methods according to embodiments of the present invention;

FIG. 9 shows a temperature graph of the two opposing microarray surfaces (S1 and S2) in a typical hybridization reaction as run by the invention.

FIG. 10 shows and alternative embodiment of the bi-thermal hybridization temperature cycle, in which the hybridization temperature (T_{H}) ramps from a higher temperature (T_{HS}) at the start of the cycles to a lower temperature (T_{HE}) at the end of the cycles.

FIG. 11 shows and alternative embodiment of the bi-thermal hybridization temperature cycle, in which the hybridization temperature for microarray S1 (T_{HS1}) is at a higher temperature than the hybridization temperature for microarray S2 (T_{HS2).}

FIGS. 12 through 15 are simplified system temperature diagrams according to the methods of FIGS. 8 and 9. It should be understood that the specific temperatures are provided as example but that in use the temperatures are defined by the user for optimal performance of the specific reaction being run. FIG. 12 is an example of system temperatures at the initiation of a hybridization reaction. FIG. 13 is an example of system temperatures during the hybridization reaction for the condition of hybridization on Surface 1, and wash condition on Surface 2. FIG. 14 is an example of system temperatures during the hybridization reaction for the condition of hybridization on Surface 2, and wash condition on Surface 1. FIG. 15 is an example of system temperatures at the end of the hybridization reaction with wash or anneal temperatures on both Surface 1 and Surface 2.

FIG. 16 shows the degree of hybridization achieved via hybridization under constant and homogeneous temperature reaction conditions (squares) versus use of the invented method using a bi-thermal reaction chamber cycled alternately between hybridize and wash on each of the array surfaces. Note that the bi-thermal hybridization reaction achieves completion in under 1 hour while iso-thermal hybridization requires ˜12-16 hours. Also note that bi-thermal hybridization results in nearly 95% hybridization while iso-thermal hybridization achieves only 35% hybridization.

FIG. 17 shows the distribution of probe between hybridization to the desired target spot versus non-specific hybridization/sticking to the array surface (background) versus probe remaining in solution at the end of an 18-hour reaction. Note that the bi-thermal reaction yields nearly 95% hybridization of probe to the desired target spot with less than 3% stuck to the rest of the slide as background and ˜2%. In comparison, note that isothermal reaction yields about 35% hybridization of probe to the desired target spot with less than 25% stuck to the rest of the slide as background and 40% left in solution.

### DETAILED DESCRIPTION OF THE INVENTION

According to the present invention, techniques including methods and systems for processing materials are provided. More particularly, the invention provides technique including methods and systems for processing for diagnostics using molecular association. Merely by way of example, the invention is applied to molecular association for diagnostics using biological arrays of materials on arrays of spots on substrates. But it would be recognized that the invention could also be applied to other molecular associative reactions such as enzyme/substrate assays, catalyst/substrate reactions, reactive chemical reactions and antibody/antigen reactions.

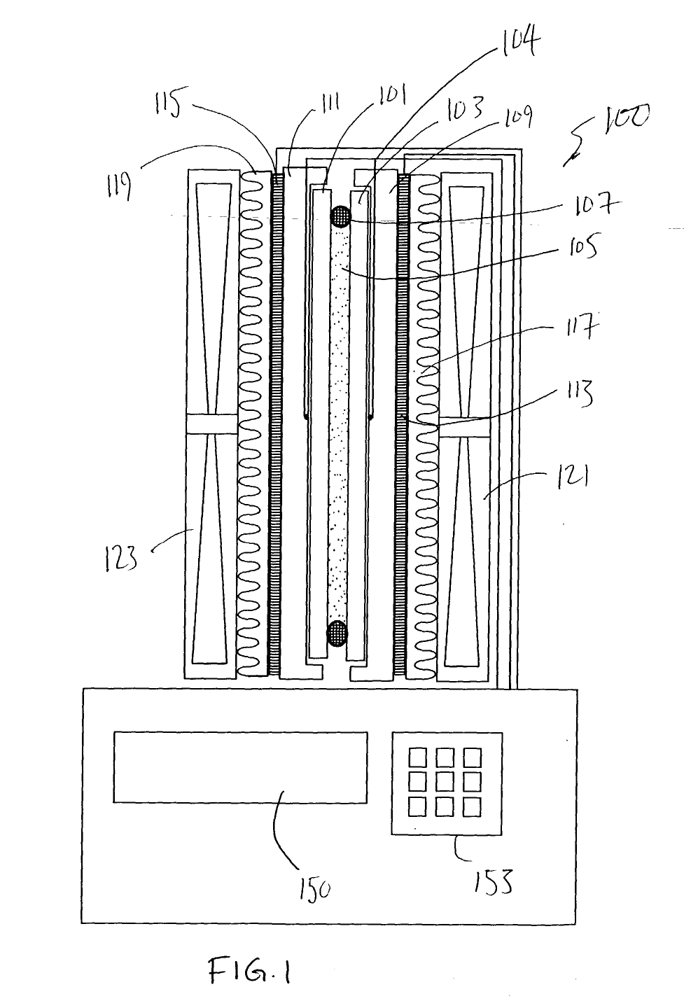

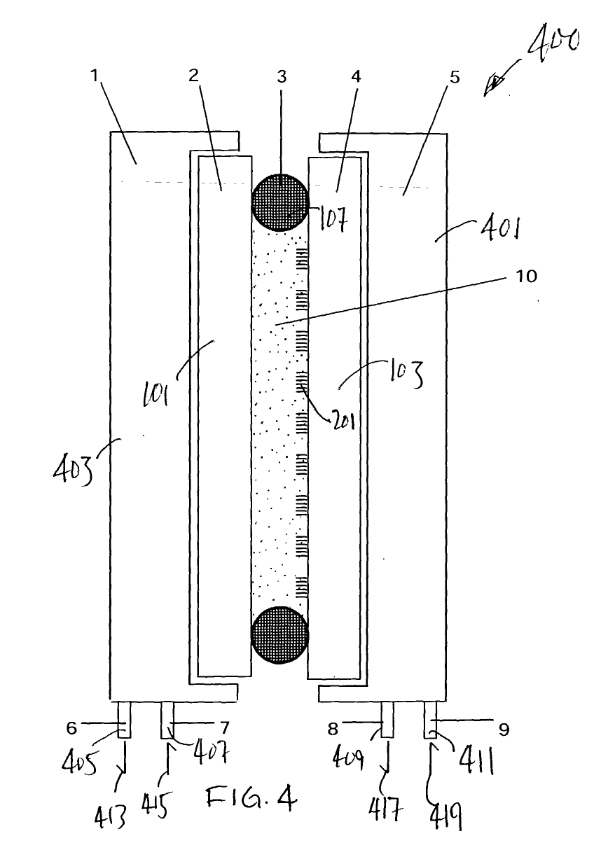

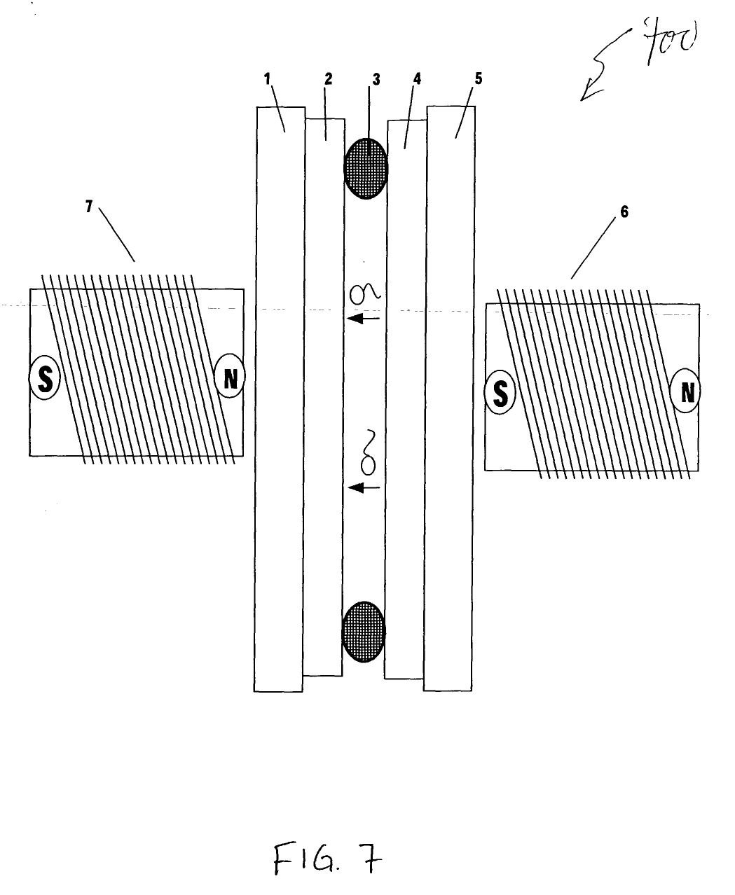

FIG. 1 is an overall system diagram 100 according to an embodiment of the present invention. This diagram is merely an example, which should not unduly limit the scope of the claims herein. One of ordinary skill in the art would recognize many other variations, modifications, and alternatives. Examples of variations on this embodiment include modification of the means of controlling the temperature at the hybridization surface such as with the use of heat transfer fluids illustrated in FIG. 4. Another variation on the embodiment modifies the system layout, achieving bi- or multi-thermal reaction conditions by separating the reactions surfaces and letting the reaction mixture flow between reaction conditions. Yet another variation of on the embodiment takes advantage of alternative motive forces for manipulation of the local concentration of reactants as is illustrated in FIG. 7.

Referring back to FIG. 1, system 100 is used for processing biologically relevant materials for reactions using molecular association. Such materials include, but are not limited to biopolymers such as nucleic acids, proteins, lipids and carbohydrates, as well as biologically relevant chemicals such as enzyme substrates, co-factors, catalysts, etc. The system includes a first substrate (e.g., glass, silicon, plastic, diamond, nylon, quartz, coated metals, ceramics, and composites, any combination of these, and the like) 103 comprising a surface. The surface includes an array of immobilized elements comprised of at least one of the species of molecules to be associated—the “target”. These immobilized elements will be referred to as “spots” or “targets”, however the term “spots” or “targets” does not imply limitation of composition or manufacture. Each of the spots comprises biologically relevant material coupled to the substrate. The spots can include, but are not limited to biopolymers such as nucleic acids, proteins, lipids and carbohydrates, as well as biologically relevant chemicals such as enzyme substrates, ligands, co-factors, catalysts, etc. The substrate 103 is held against an assemblage such that a chamber is formed—with one surface comprised of the test surface of the first substrate. In the preferred embodiment, the chamber surface opposite the first substrate can be either another substrate with its test surface facing into the chamber, or a “blank” substrate without test surface. Alternatively to this preferred embodiment, the chamber may be merely an enclosing structure forming a chamber. The second substrate 101 is separated from the first substrate by a predetermined distance—forming the reaction chamber. Preferably, the predetermined distance ranges from about 0.1 mm to about 2 mm, but can be others. Each of the substrates also includes a surface that are substantially parallel to each other. Alternatively, each of these surfaces may be at an angle with each other. Additionally, the second substrate can be made of a similar material as the first substrate, as well as other materials. Such materials include glass, silicon, plastic, diamond, nylon, quartz, coated metals, ceramics, and composites, any combination of these, and the like.

The system also has a fluid disposed within the reaction chamber. The fluid carries molecules intended to react with the spots or targets on the test surface(s). The fluid can be selected from an inert solution and/or a reactive solution. The solution can be conductive and/or insulate. The conductive solution can be biologically relevant or useful, such as saline or buffered aqueous solutions. As merely an example, such solution can include SSC, but may be composed of other materials. The inert solution can be polymers and monomers intended to exclude volume, as well as other materials with other purposes. The fluid is bound by the seal 107, which form a pocket region or reaction chamber between the first substrate and the second substrate. The substrates 101, 103 are respectively coupled to heat transfer block 111 and 109. Each of the heat transfer blocks is firmly engaged with the substrate. The heat transfer block transfers heat to and from the substrate. The heat transfer block is made of a suitable material such as thermally conductive ceramics or metals, but can be others. The heat transfer block also has a temperature sensor 104 such as a thermocouple, thermister or infrared probe to detect a temperature of the substrate. The temperature sensor is connected to controller 150, which includes interface 153. A first energy source 113 is coupled to the first substrate to provide a first desired temperature to the first substrate. A second energy source 115 is coupled to the second substrate to provide a second desired temperature to the second substrate. The energy source can be a solid-state temperature control device such as those employing the Peltier thermo-electric effect. Each Peltier apparatus can include heat sinks 117, 119, which couple to the Peltier apparatus. As shown, the heat sinks can include fins, which allow heat to be removed from the Peltier apparatus. Further facility removal of heat, each of the heat sinks may be coupled to fans 123, 121 depending upon the embodiment.

In the preferred embodiment, the first substrate and the second substrate are arranged in manner where surfaces of each of the substrate are parallel to gravity, as shown. That is, such surfaces are maintained in vertical alignment. Fluid disposed between each of the substrates is arranged in a volume, which has a length, a width, and a thickness. The length is aligned in a vertical manner and is parallel to gravity. The thickness is the predetermined distance, which separates each of the substrates from each other. The width is normal to gravity. The length, width, and thickness define the volume of the fluid. Although the present substrates are arranged in a trapezoidal volume, there can be many other shapes, e.g., annular. Of course, one of ordinary skill in the art would recognize many other variations, modifications, and alternatives.

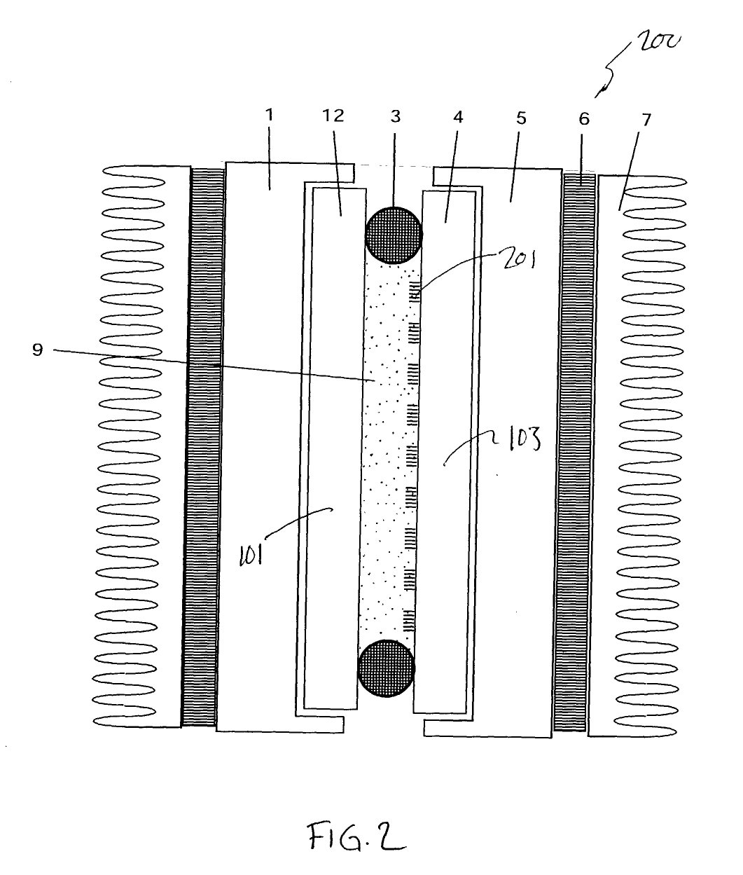

FIG. 2 is a more detailed side-view diagram of a system 200 according to an embodiment of the present invention. This diagram is merely an example, which should not unduly limit the scope of the claims herein. One of ordinary skill in the art would recognize many other variations, modifications, and alternatives. Like reference numerals are used in this diagram as some of the others, but are not intended to be limiting. As shown, substrate 103 includes spots 201. Each of the spots is a biological material such as nucleic acids, proteins, lipids and carbohydrates, but can be others. Substrate 101 may either be another test substrate with spots facing into the reaction chamber, or may be a simple blank. When resources are determined more by the substrate surface costs, or when two tests are not required, the second surface can be a blank. As merely an example, a top-view diagram of the spots, including substrate, is provided in the simplified diagram of FIG. 2A.

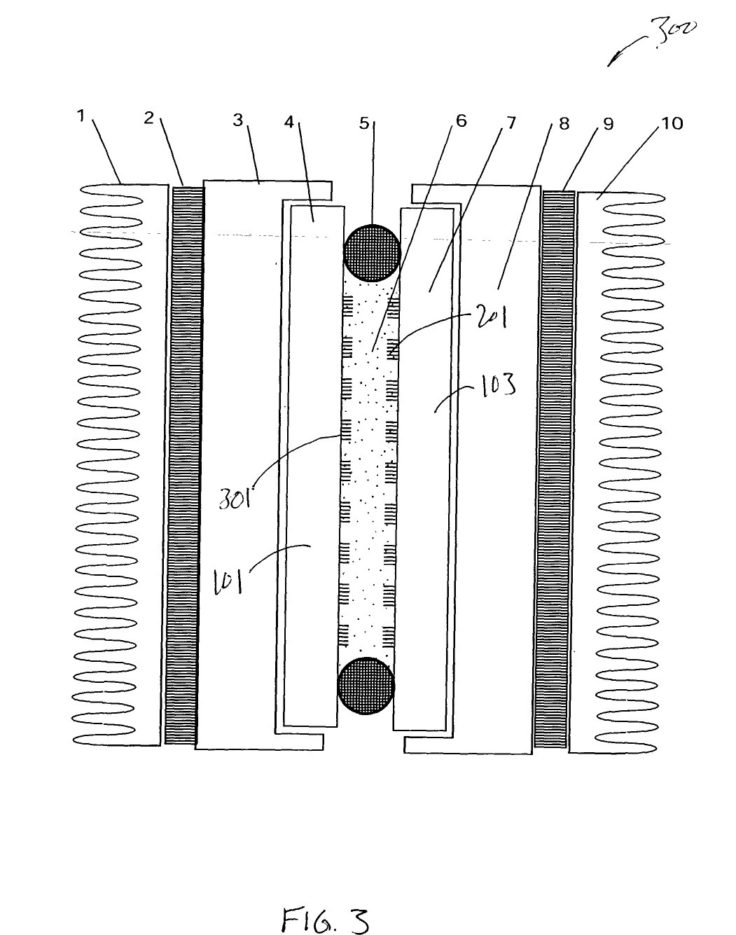

FIG. 3 is a simplified side-view diagram of a system 300 according to an alternative embodiment of the present invention. This diagram is merely an example, which should not unduly limit the scope of the claims herein. One of ordinary skill in the art would recognize many other variations, modifications, and alternatives. As shown, substrate 103 includes spots 201. Each of the spots is a biologically relevant such as biopolymers such as nucleic acids, proteins, lipids and carbohydrates, as well as biologically relevant chemicals such as enzyme substrates, co-factors, catalysts, etc., but can be others. Substrate 101 includes spots 301. When cost of the tests are determined be the costs of the “probe” material (fluid) against which the test substrates react, two opposing test substrates may be employed to better utilize the reaction fluid.

Additionally, the use of a second test substrate can allow for greater reliability and confidence in the test, while using fewer resources than performing two completely separate tests. Further, the reaction conditions for the two opposite surfaces of the reaction chamber can differ in such a way (i.e. different temperatures) as to provide significant new information in the molecular association reaction.

FIG. 4 is a simplified side-view diagram of an alternative system 400 according to an embodiment of the present invention with temperature regulation achieved through the use of cooling or heating fluids or through the use of electrical resistive heating rather than Peltier effect thermoelectric temperature controllers. Like reference numerals are used in this diagram as some of the previous diagrams, which are not intended to be limiting. As shown, the system includes substrate 103 and substrate 101. A plurality of spots 201 are also included. Each of the substrates is separated by a predetermined distance, which is separated by seals 107. Each of the substrates includes heat sink 401 and 403 respectively. Each of the heat sinks includes incoming 411 and outgoing 409 fluid lines.

As shown, incoming fluid 419 enters line 411 traverses through heat sink 401 and exits 417 via line 409. Similarly, incoming fluid 415 enters line 407 traverses through heat sink 403 and exits 413 via line 405. As merely an example, FIG. 4 illustrates a cross-sectional view of the heat sink according to an embodiment of the present invention. Alternative methods controlling the temperature of have advantages of achieving greater heating and cooling rates, in achieving greater operating temperature ranges, and in achieving greater temperature differentials between the two chamber test surfaces. Additionally, alternative methods controlling chamber surface temperatures may better fit the economic demands of a specific application.

A method for operating the present system for mixing fluid is provided as follows:

1. Provide system;

2. Maintain fluid between first and second substrate;

3. Apply first temperature to first substrate;

4. Apply second temperature to second substrate, where the second temperature is different from the first temperature;

5 Maintain a predetermined temperature difference between the first and second substrate;

6. Drive fluid to circulate through volume defined between the first substrate and the second substrate;

7. Maintain fluid circulation;

8. Adjust temperature if desired; and

9. Perform other steps, as desired.

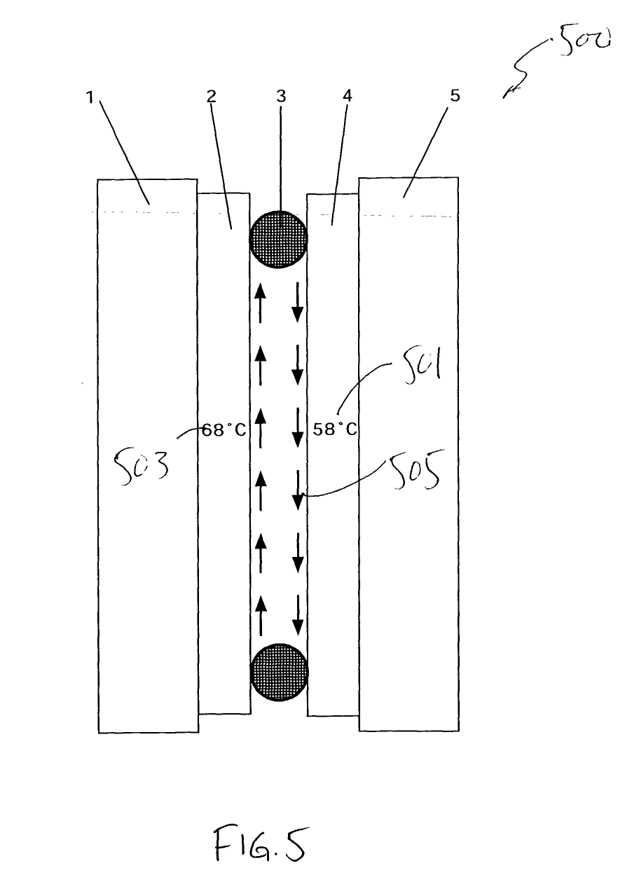

FIG. 5 illustrates the mixing effect 500 achieved with a temperature differential between zones of the reaction chamber. Another variation on the embodiment modifies the system layout, achieving bi- or multi-thermal reaction conditions by separating the reactions surfaces and letting the reaction mixture flow between reaction conditions. As shown, the system-includes a first substrate and a second substrate, which are separated with a predetermined distance from each other. Fluid is maintained between first and second substrates. The system applies a first temperature 501 to the first substrate and applies a second temperature 503 to the second substrate, where the second temperature is different from the first temperature. Preferably, the temperature is predetermined and is at least 5 degrees centigrade for applications involving the molecular association of nucleic acids through hybridization. The temperature difference drives fluid to circulation 505 through volume defined between the first substrate and the second substrate, as shown. The system maintains fluid circulation and may also adjust the temperature difference if desired.

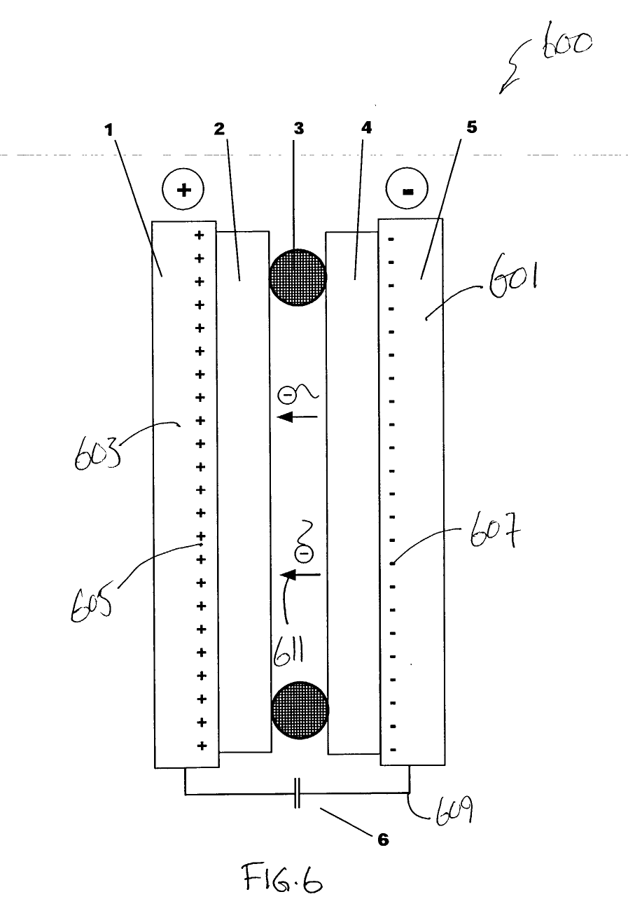

FIG. 6 illustrates a system 600 including local concentration control achieved with a motive force on reactants (electric, magnetic or density driven) according to an embodiment of the present invention. Each of the temperature regulation plates 601, 603 is coupled to a voltage difference 605, 607. An energy source 609 is coupled between each of the plates. Electric charge 605 is applied to the temperature regulation plates driving reactants away 611 from the plate with like charge and towards the plate with opposite charge. As merely an example, biological materials such as DNA is often electrically charged, which allows for independent control of concentration. Depending upon the embodiment, there can be many alternatives, modifications, and variations.

FIG. 7 illustrates a system 700 including local concentration control achieved with a motive force on reactants according to an alternative embodiment of the present invention. In this alternative embodiment, a magnetic field is generated such that paramagnetic reactants can be driven towards either, or charged reactants can be driven perpendicular to a fluxating magnetic field. Direction of the motive force may be controlled by controlling the polarity of electricity flowing through the electromagnets. In yet another alternative embodiment, reactants may be driven to desired locations based on density using gravity or centrifugation.

In a specific embodiment, the present systems of FIGS. 6 and 7 are used in methods according to embodiments of the present invention. Such methods control reactant concentration in a volume independent fashion. Volume independent concentration control is especially useful and desirable for bi-thermal reactions such as hybridization. With the present system, volume independent concentration control not only reduces inhomogeneity problems without reducing reaction rate, but also is employed to help cycle reactants from low temperature conditions to high according to some embodiments.

Further volume independent concentration control can be used to facilitate the wash/quantify steps of processing a microarray. Correctly hybridized material will remain on the chip even under external electrophoretic, magnetic or gravitational pressure to move away from the array. Incorrectly hybridized material rapidly separates from the microarray under external electrophoretic, magnetic or gravitational pressure, increasing the effective wash rate, reducing background, and enabling direct quantification even without removal of the reaction solution.

Electrophoresis is commonly used in molecular biology to move DNA through gels. The gels retard movement in a size dependant manner—letting one get a measurement of the size of a DNA fragment. The gel is generally a slab bathed in a saline solution. When a voltage of ˜5-10 V/cm of gel is applied, the negatively charged DNA moved towards the positive end, covering ˜2 cm/hour (though small fragments faster and larger fragments slower).

In the preferred embodiment, we have employed electrophoresis to drive the probe DNA from one side of present system chamber to the other DNA. We have also provided design for achieving the same effects using magnetic or gravitational/centrifugal based motive force on the reactants to be concentrated.

In the preferred embodiment, a reaction chamber is contained by two opposing microscope slides (#2 and #4—FIG. 7), which sandwich an o-ring (#3—FIG. 7). The reaction chamber inside the o-ring is ˜0.5 mm across/deep, and 20 cm on each side. These dimensions are only given as example, with changes in dimensions to accommodate other array sizes or reaction volumes understood to be possible.

We concentrate the probe cDNA probe at the surface of one of the slides, then move it across to the other and back again using electrostatic force. This is achieved by using two opposing conductive plates (#1 & #5—FIG. 7) which sandwich the microarray bearing slides (#2 & #4—FIG. 7). The plates can be held at any specific voltage (#6—FIG. 7). Controlling the electrophoretic voltage enables control on the degree of concentration achieved and the speed of achieving this concentration.

Typically, we use a voltage differential between 20 and 600 volts, however higher or lower voltage differentials are useful for some applications. Charged molecules within the reaction chamber will experience a motive force—pushing them away from the similarly charged plate and towards the oppositely charged plate.

The electromotive force concentrating the cDNA probe acts against thermal motion diffusing it. After a period of time determined by the eletrophoretic voltage, viscosity of the reaction solution, dimensions of the reaction chamber, and characteristic of the cDNA probe, the use of an electrophoretic voltage results is a steady-state reactant concentration profile that is determined by the opposing forces of electrophoresis and diffusion.

The voltage differential is oriented and applied such that the array undergoing hybridization is exposed to probe cDNA at concentrations far higher than would be expected if the probe cDNA were evenly distributed in the reaction solution. Further, the voltage can be alternated such that for a portion of each hybridization cycle, the probe CDNA cycles between being concentrated in the higher temperature region of the reaction chamber, and the lower temperature region of the reaction chamber.

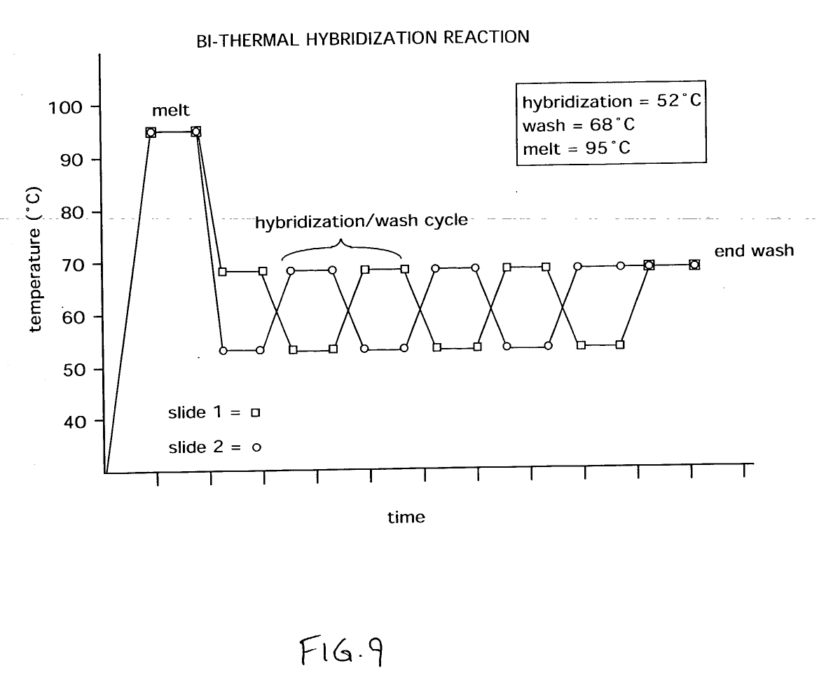

In the preferred embodiment, a 230-volt bias is applied to the two opposing conductive plates (FIG. 7), which sandwich the microarray bearing slides (FIG. 7). Using temperature programs of either, constant or ramped temperature endpoints (which are further described in FIGS. 9-11), the electrophoretic charge bias was applied such that reactants moved away from the lower temperature (hybridization) side during the first half of each cycle, and towards the lower temperature (hybridization) side during the last half of each cycle.

The above embodiments describe aspects of the invention illustrated by elements in simplified system diagrams. As will be understood by one of ordinary skill in the art, the elements can be implemented in computer software. The elements can also be implemented in computer hardware. Alternatively, the elements can be implemented in a combination of computer hardware and software. Some of the elements may be integrated with other software and/or hardware, or specialized hardware (e.g. an ASIC). Alternatively, some of the elements may be combined together or even separated. It is also understood that the examples and embodiments described herein are for illustrative purposes only and that various modifications or changes in light thereof will be suggested to persons skilled in the art and are to be included within the spirit and purview of this application and scope of the appended claims. Further details of methods according to embodiments of the present invention are provided as follows.

A method for processing biological materials for diagnostics according to an embodiment of the present invention is outlined as follows:

1. Provide apparatus for processing, including a first substrate and a second substrate;

2. Dispose fluid between the first substrate and the second substrate;

3. Maintain fluid immersing a surface including an array of spots of the first substrate;

4. Maintain a physical separation between the first substrate and the second substrate by a predetermined distance, whereupon the fluid occupies the predetermined distance and overlies each of the spots in the array;

5. Apply a first energy to the first substrate;

6. Cause a first desired temperature to the first substrate using the first energy;

7. Apply a second energy to the second substrate;

8. Cause a second desired temperature to the second substrate using the second energy;

9. Form a temperature gradient between the first substrate and the second substrate through the fluid occupied within the predetermined distance;

10. Enhance using the first temperature a first reaction process at the array of spots; and

11. Enhance using the second temperature a second reaction process at the second substrate.

The above sequence of steps provides a method for processing biological materials using application of energy via the first substrate and the second substrate. The energy forms a temperature gradient between the substrates, which also causes fluid convective forces. Such convective forces also assists with the processing in preferred embodiments. These and other details of the present method are provided throughout the present specification and more particularly below.

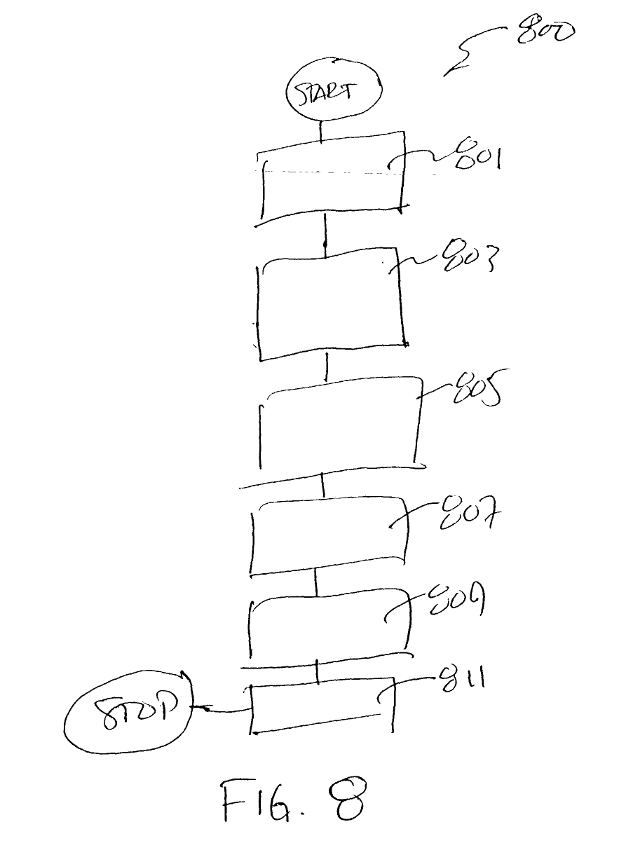

FIG. 8 is a simplified flow diagram of methods 800 according to embodiments of the present invention. This diagram is merely an example, which should not unduly limit the scope of the claims herein. One of ordinary skill in the art would recognize many other variations, modifications, and alternatives. As shown, the method includes providing (step 801) an apparatus for processing, including a first substrate and a second substrate. Such apparatus can be similar to the ones provided above as well as outside of this specification. The method disposes (step 801) fluid between the first substrate and the second substrate. Each of the substrates can be made of a suitable material. For example, such materials include glass, silicon, plastic, diamond, nylon, quartz, coated metals, ceramics, and composites, any combination of these, and the like.

The method maintains (step 803) fluid immersing a surface including an array of spots of the first substrate. The method also maintains (step 805) a physical separation between the first substrate and the second substrate by a predetermined distance (e.g., 0.01 to 0.5 millimeters). The fluid occupies the predetermined distance and overlies each of the spots in the array. The method applies (step 807) a first energy to the first substrate. Preferably, the first energy causes a first desired temperature to the first substrate. The method applies (step 809) a second energy to the second substrate. Preferably, the second energy causes a second desired temperature to the second substrate. The method forms (step 811) a temperature gradient between the first substrate and the second substrate through the fluid occupied within the predetermined distance. The first temperature enhances a first reaction process at the array of spots and the second temperature enhances the second temperature a second reaction process at the second substrate. Depending upon the embodiment, the first temperature increases a reaction rate or reduces a reaction rate of species on one or more of the spots. Additionally, the second temperature increases a reaction rate or reduces a reaction rate of species on one or more of the spots. Additionally, the method forms a temperature gradient between the substrates, which also causes fluid convective forces to promote movement of the fluid. Such convective forces also assists with the processing in preferred embodiments. Of course, one of ordinary skill in the art would recognize many other variations, modifications, and alternatives.

Additionally, the above sequence of steps is performed using a combination of hardware and software. These steps can be further combined or even separated in computer software. Additionally, these steps can be further combined or even separated in computer hardware. The steps can also be combined with any combination of hardware and/or software, depending upon the embodiment. Accordingly, the present method is not intended to be limiting with respect to the type of technology that is presently available.

FIG. 9 shows a temperature graph of the two opposing microarray surfaces (S1 and S2) in a typical hybridization reaction as run by the invention. It should be understood that the specific temperatures are provided as example but that in use the temperatures are defined by the user for optimal performance of the specific reaction being run. This diagram is merely an example, which should not unduly limit the scope of the claims herein. One of ordinary skill in the art would recognize many other variations, modifications, and alternatives.

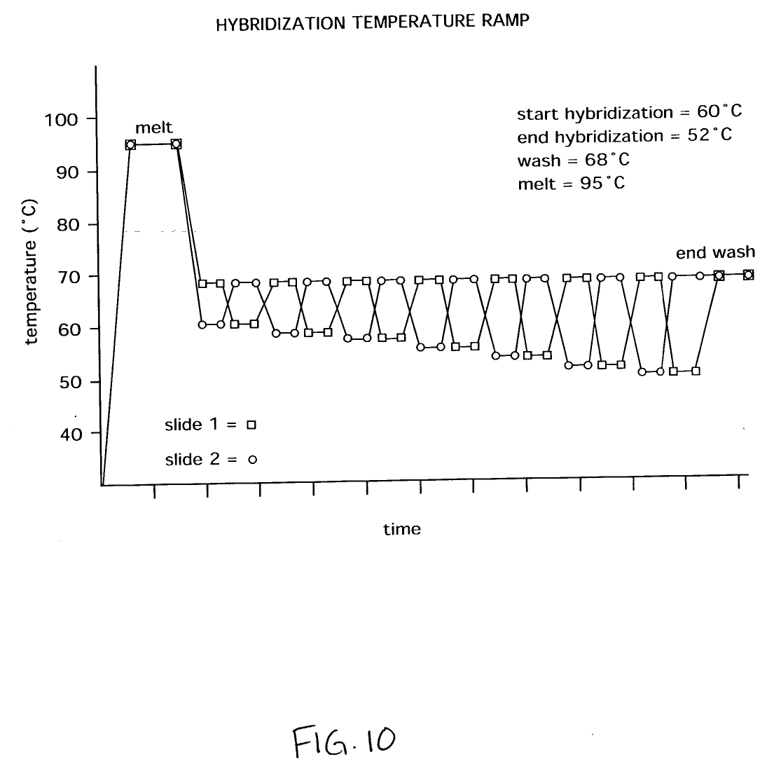

FIG. 10 shows and alternative embodiment of the bi-thermal hybridization temperature cycle, in which the hybridization temperature (T_{H}) ramps from a higher temperature (T_{HS}) at the start of the cycles to a lower temperature (T_{HE}) at the end of the cycles. It should be understood that the specific temperatures are provided as example but that in use the temperatures are defined by the user for optimal performance of the specific reaction being run. This diagram is merely an example, which should not unduly limit the scope of the claims herein. One of ordinary skill in the art would recognize many other variations, modifications, and alternatives.

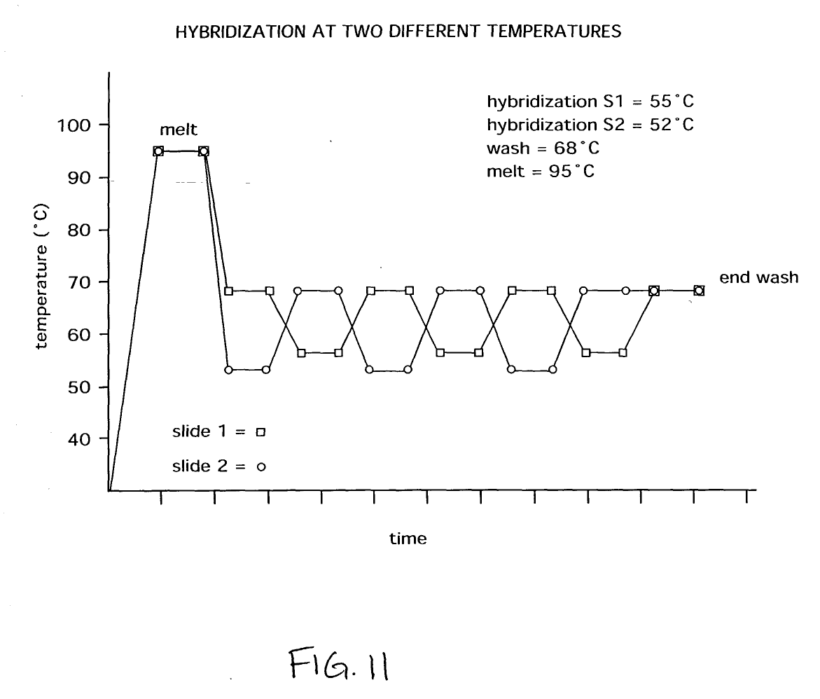

FIG. 11 shows and alternative embodiment of the bi-thermal hybridization temperature cycle, in which the hybridization temperature for microarray S1 (T_{HS1}) is at a higher temperature than the hybridization temperature for microarray S2 (T_{HS2}). It should be understood that the specific temperatures are provided as example but that in use the temperatures are defined by the user for optimal performance of the specific reaction being run. This diagram is merely an example, which should not unduly limit the scope of the claims herein. One of ordinary skill in the art would recognize many other variations, modifications, and alternatives

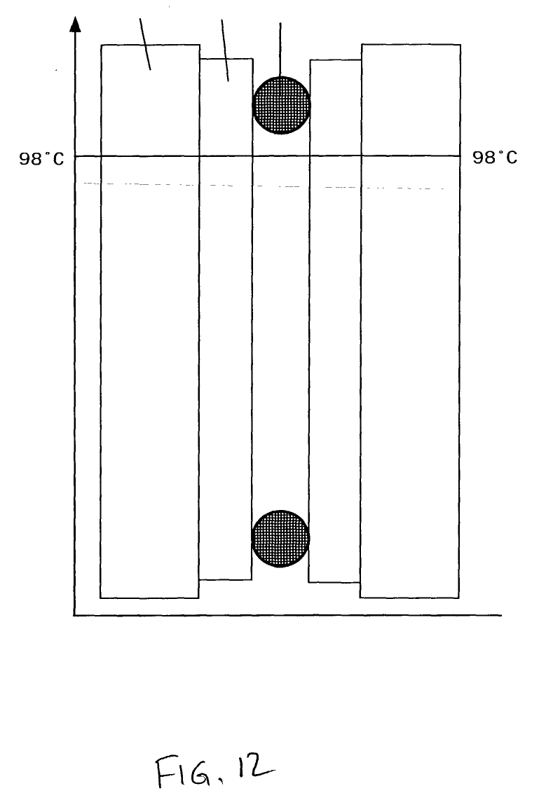

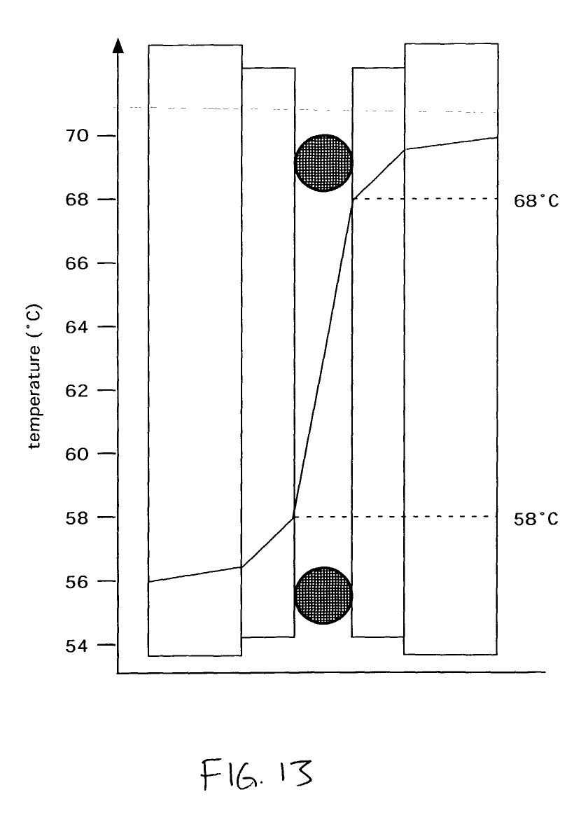

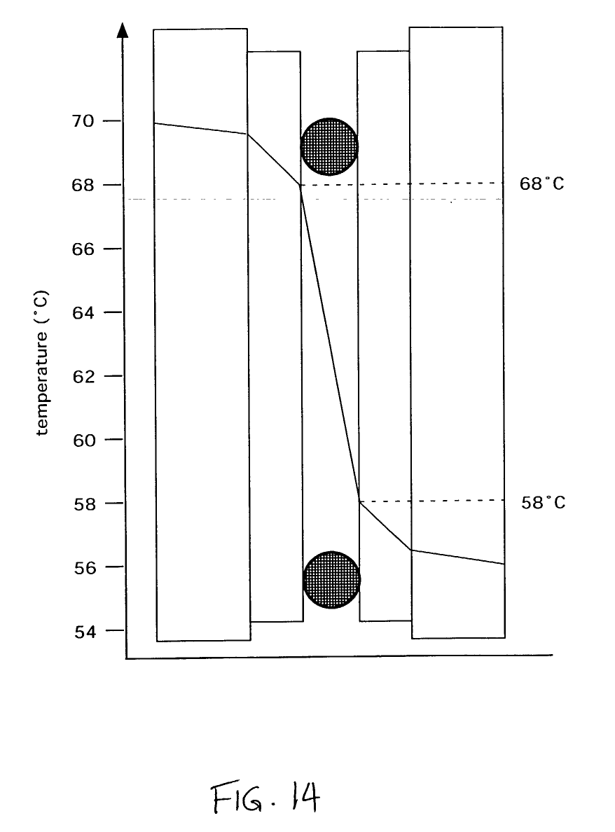

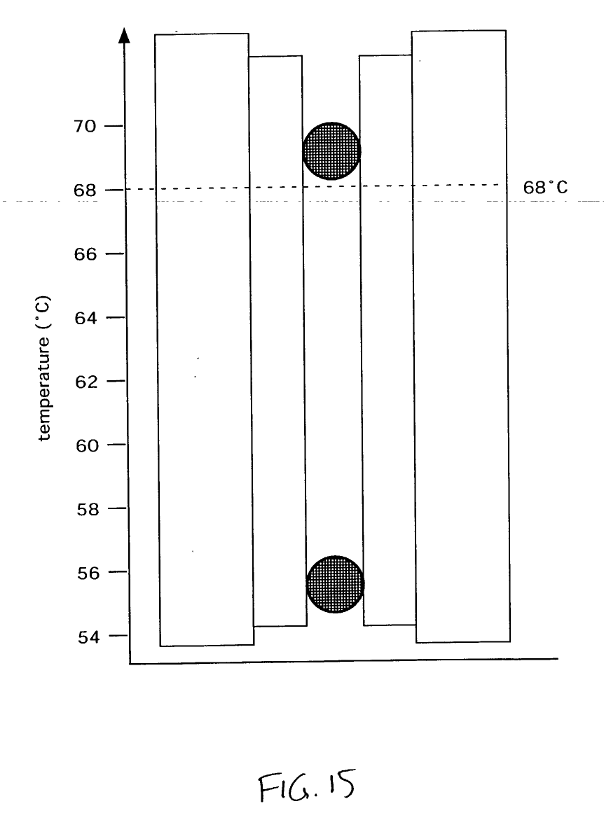

FIGS. 12 through 15 are simplified system temperature diagrams according to the methods of FIGS. 8 and 9. It should be understood that the specific temperatures are provided as example but that in use the temperatures are defined by the user for optimal performance of the specific reaction being run. FIG. 12 is an example of system temperatures at the initiation of a hybridization reaction. FIG. 13 is an example of system temperatures during the hybridization reaction for the condition of hybridization on Surface 1, and wash condition on Surface 2. FIG. 14 is an example of system temperatures during the hybridization reaction for the condition of hybridization on Surface 2, and wash condition on Surface 1. FIG. 15 is an example of system temperatures at the end of the hybridization reaction with wash or anneal temperatures on both Surface 1 and Surface 2. Once again, these diagrams are merely examples, which should not unduly limit the scope of the claims herein. One of ordinary skill in the art would recognize many other variations, modifications, and alternatives.

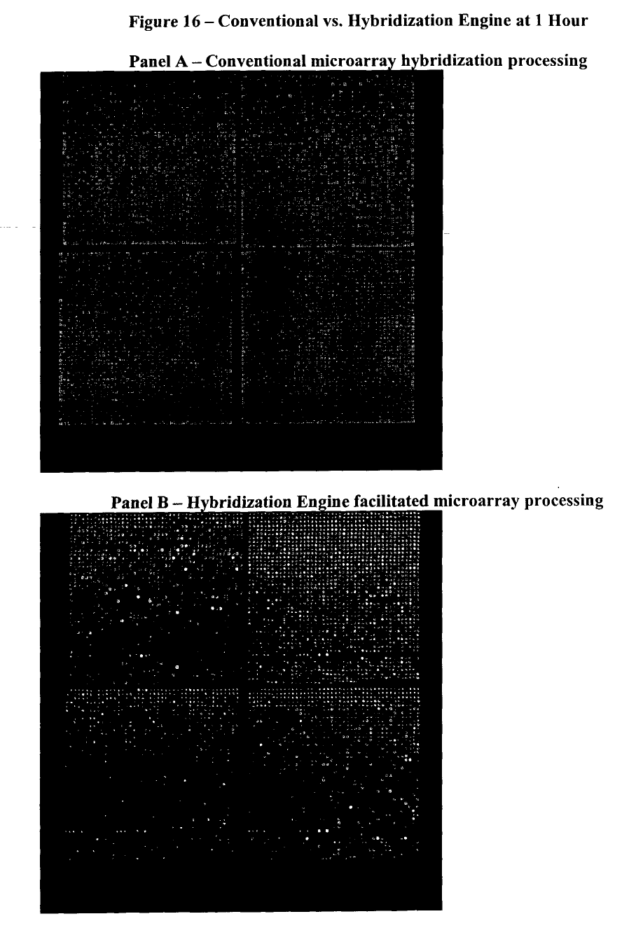

FIG. 16 shows the degree of hybridization achieved via hybridization under constant and homogeneous temperature reaction conditions (squares) versus use of the invented method using a bi-thermal reaction chamber cycled alternately between hybridize and wash on each of the array surfaces. Note that the bi-thermal hybridization reaction achieves completion in under 1 hour while iso-thermal hybridization requires ˜12-16 hours. Also note that bi-thermal hybridization results in nearly 95% hybridization while iso-thermal hybridization achieves only 35% hybridization.

Hybridization under standard (isothermal) or hybridization engine (bi-thermal) conditions show clear advantage to bi-thermal hybridization. Microarray data is usually normalized for total brightness to facilitate comparison between different hybridizations. In this figure Panel A and Panel B have been normalized for total brightness. This normalization enhances the brightness of Panel A (conventional). This alone suggests hybridization in Panel B (Hybridization Engine) is better. With normalization, one can be seen is that hybridization is considerably better in Panel B (Hybridization Engine). This is seen in that differences in gene expression (red verses green indications) are clearly discernable in Panel B, while these differences are undetectable in Panel A (conventional).

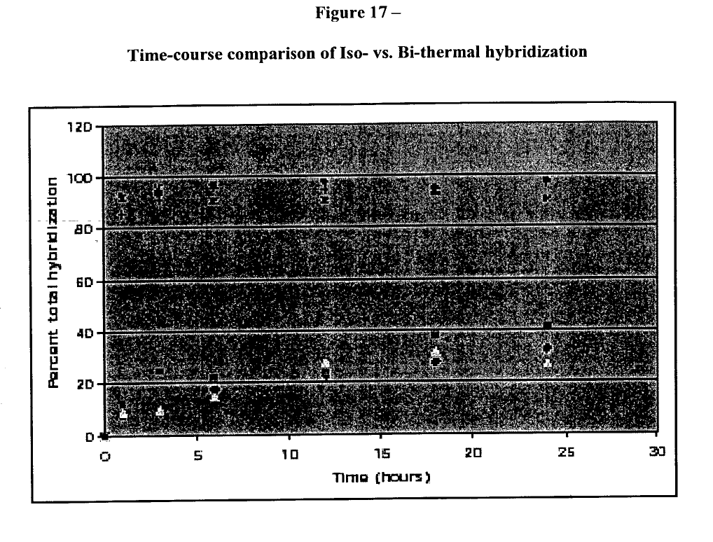

FIG. 17 shows the distribution of probe between hybridization to the desired target spot versus non-specific hybridization/sticking to the array surface (background) versus probe remaining in solution at the end of an 18-hour reaction. Note that the bi-thermal reaction yields nearly 95% hybridization of probe to the desired target spot with less than 3% stuck to the rest of the slide as background and ˜2%. In comparison, note that isothermal reaction yields about 35% hybridization of probe to the desired target spot with less than 25% stuck to the rest of the slide as background and 40% left in solution.

Hybridization under standard (iso-thermal) or hybridization engine (bi-thermal) conditions was quantified for time points of 0, 3, 6, 12, 18, and 24 hours. Three samples were tested for each time point and each condition. It can be seen that hybridization under conventional isothermal conditions results in slow hybridization reaching close to 40% at 24 hours. Bi-thermal hybridization facilitated by the Hybridization Engine quickly reached near 90% hybridization by 1 hour.

Hybridization under standard (iso-thermal) or hybridization engine (bi-thermal) conditions was quantified for time points of 0, 3, 6, 12, 18, and 24 hours. Three samples were tested for each time point and each condition. It can be seen that hybridization under conventional isothermal conditions results in slow hybridization reaching close to 40% at 242 hours. By-thermal hybridization facilitated by the Hybridization Engine quickly reached near 90% hybridization by 1 hour.

Data for FIG. 17.

Hybridization Condition

| | |
|---|---|
| | Bi-thermal |
| Iso-Thermal | (Hybridazation Engine) |

| Time (Hours) | | | | Average | | | | Average |
|---|---|---|---|---|---|---|---|---|
| 0 | 0 | 0 | 0 | 0.0 | 0 | 0 | 0 | 0.0 |
| 1 | 8 | 12 | 9 | 9.7 | 92 | 93 | 86 | 90.3 |
| 3 | 9 | 25 | 10 | 14.7 | 96 | 94 | 91 | 93.7 |
| 6 | 18 | 22 | 15 | 18.3 | 91 | 97 | 98 | 94.7 |
| 12 | 23 | 24 | 28 | 25.0 | 91 | 98 | 95 | 94.7 |
| 18 | 38 | 38 | 32 | 32.7 | 94 | 95 | 94 | 94.3 |
| 24 | 33 | 41 | 27 | 33.7 | 91 | 98 | 97 | 95.3 |

Here we disclose invention of a basic design paradigm for instruments intended to facilitate molecular associations. Additionally we disclose embodiments if this design paradigm demonstrating generalization of the principle and a range of applications for the invention. Specifically, this invention describes a new method of controlled use of non-homogeneous reaction temperatures to can increase the speed and efficiency of molecular association, as well as reduce wastage of reaction components through degradation or inappropriate reaction. A number of types of bio-molecular association can be facilitated with this invention, including antibody/antigen association, enzyme/substrate association, receptor/ligand association, and nucleic acid hybridization. Other types of molecular association such as reaction components or reaction components and catalyst can also be significantly improved by means of this invention.

The invention disclosed here is the use of independently temperature controlled reaction regions within a reaction mix to 1) allow of optimal temperature for molecular association while simultaneously allowing for maintenance of unassociated reactants at a preferred temperature. Use of different temperatures in a reaction chamber also allows for convection driven mixing. This invention is useful in cases where the optimal temperature for molecular association is different from the optimal temperature for maintaining unassociated reactants. This invention is also useful in situations where thermal convection driven flow or mixing is desirable (e.g. to help bring reactants together or to reduce overexposure of reactants to association surface).

One of ordinary skill in the art would recognize many other variations, modifications, and alternatives. The above example is merely an illustration, which should not unduly limit the scope of the claims herein. One of ordinary skill in the art would recognize many other variations, modifications, and alternatives. It is also understood that the examples and embodiments described herein are for illustrative purposes only and that various modifications or changes in light thereof will be suggested to persons skilled in the art and are to be included within the spirit and purview of this application and scope of the appended claims.

Experimental Results:

To prove the principle and operation of the present invention, experiments have been performed to quantify hybridization kinetics and yields for conventional isothermal reaction conditions and compared to the invention's use of bi- or multi-thermal reaction conditions. These experiments are reported merely as illustrative examples of the performance benefits derived by the invention for molecular association reactions. These examples should not unduly limit the scope of the claims herein.

In this experiment, the system of FIG. 1 was used for both isothermal and bi-thermal reactions. For isothermal reactions, the temperatures of both opposing microarray surfaces were identical. For bi-thermal reactions, the temperature at S1 and S2 varied according to FIG. 9. 3-minute cycles were used with a hybridization temperature of 65° C. and a wash temperature of 75° C. Hybridization solutions consisted of yeast cDNA spiked with Cy5 labeled CUP1 CDNA and Cy3 labeled ACT1 in 3×SSC, 0.2% SDS and polyA-DNA (See labeling protocol below). Cy5 and Cy3 spike CDNA was added to 1/10 the molar quantity of target CDNA spotted on microarrays. Cy5 and Cy3 labeled DNA was purified away from unincorporated Cy5 and Cy3. Cy5 and Cy3 labeled DNA was quantified by total Cy5 or Cy3 florescence.

I. Preparation of Cy3 and Cy5 Labeled of Probes

| | Cy3 rxn | | Cy5 rxn DNA | |
|---|---|---|---|---|
| (2 ug PCR product) | 12.9 | (ACT1) | 12.9 | (CUP1) |
| Single side priming oligo (2 ug/uL) | 2.5 | | 2.5 | |
| | 15.4 | uL | 15.4 | uL |

1. Heat 10 min. {fourth root}70C. Quick chill on ice.

2. PREPARE POLYMERASE COCKTAIL FOR BOTH RXNS:

| | X 1 | | X 2.5 | | Final conc. |
|---|---|---|---|---|---|
| 5X Polymerase buffer | 6.0 | | 15.0 | | 1X |
| DTT (0.1 M) | 3.0 | | 7.5 | | 10 mM |
| 50X dNTPs | 0.6 | | 1.25 | | 500 uM; |
| | | | | | 200 uM dTTP |
| Cy3- or Cy5 dTTP | 3.0 | | — | | 100 uM |
| Klenow (200 U/uL) | 2.0 | | 5.0 | | 13 U/uL |
| | 14.6 | uL | 11.6 | uL | aliquots |

3. Add 3.0 uL Cy3 or Cy5 to respective primer-annealed DNA's. Aliquot 11.6 uL of polymerase cocktail to each rxn for total volume of 30 uL. Incubate 2 hr @42C. Place on ice.

4. Place 500 uL TE (pH 7.4) each in two microcon-30 filters. Add RT rxns to each microcon filter. Centrifuge 7 min. at top speed. Repeat TE washes 2 times, or until all unincorporated dye is removed.

5. Inspect filters. Centrifuge in 30 sec. intervals until volume is 10-20 uL.

6. Invert filters into fresh tubes. Centrifuge 1 min. to harvest labeled DNA's.

7. Quantify DNA in Hoffer DNA Fluorometer (Model TKO 100).

8. Dilute DNA's to 1/10^{th} molar quantity of target spot DNA.

9. Mix together Cy3- and Cy5-labeled DNA's with unlabeled yeast cDNA's. Concentrate sample to ˜10-12 uL using microcon filter or vacuum pump.

10. Add 1 uL polyA DNA or RNA for non-specific hybridization. Add 3 uL 20×SSC for total volume of 12-15 uL.

11. Pre-wet millipore filter by adding 5 uL ddH20. Centrifuge 1 min. at top speed. Remove eluted water with pipet tip.

12. Add probe to filter. (Pipet probe onto filter wall, not directly onto membrane.) Centrifuge 1 min. at top speed.

II. Hybridizing the Cy3 and Cy5 Labeled Probes on Microarrays

1. Place microarray S1 on temperature control plate T1.

2. Apply hybridization chamber sealing gasket around microarray.

3. Place the entire probe volume on the array framed by hybridization chamber sealing gasket.

4. Overlay opposing microarray S2.

5. Overlay temperature control plate T2 and Temperature control assembly.

6. Fasten sealing mechanism.

7. Hybridize according to appropriate temperature regimen.

a. For isothermal reactions, the temperature of both opposing microarray surfaces were identical.

b. For bi-thermal reactions, the temperature at S1 and S2 varied according to FIG. 9. 3-minute cycles were used with a hybridization temperature of 65° C. and a wash temperature of 75° C. For bi-thermal reactions, the microarray assembly was rotated such that the long axis of the microarray slide stands vertically.

II. Collection of Post-process Hybridization Mix:

1. With hybridization reaction idled at wash or anneal temperature for both S1 and S2, prepare hybridization unit for removal of slides. (Rotate back to horizontal if vertical). Prepare first wash (2×SSC, 0.03% SDS—see #1A in section below).

2. Remove fasteners and carefully remove top temperature control unit (T1).

3. Remove top microarray (S2). Place it in wash.

4. Collect hybridization solution with pipette and place in 0.65 ml tube.

5. Remove chamber sealing gasket and place bottom microarray (S1) in wash.

III. Washing and Scanning Arrays

1. Ready washes in 250 ml chambers to 200 ml volume as indicated in the table below. Avoid adding excess SDS. The Wash 1A chamber and the Wash 2 chambers should each have a slide rack ready. All washes are done at room temperature.

2. Wash microarrays as follows:

| Wash | Description | Vol (ml) | SSC | SDS (10%) |
|---|---|---|---|---|
| 1A | 2x SSC, 0.03% SDS | 200 | 200 ml 2x | 0.6 ml |
| 1B | 2x SSC | 200 | 200 ml 2x | — |
| 2 | 1x SSC | 200 | 200 ml 1x | — |
| 3 | 0.2x SSC | 200 | 200 ml 0.2x | — |

3. Blot dry chamber exterior with towels and aspirate any remaining liquid from the water bath.

4. Unscrew chamber; aspirate the holes to remove last traces of water bath liquid.

5. Place arrays, singly, in rack, inside Wash I chamber (maximum 4 arrays at a time). Allow cover slip to fall. Agitate for 2 min.

6. Remove array by forceps, rinse in a Wash II chamber without a rack, and transfer to the Wash II chamber with the rack. This step minimizes transfer of SDS from Wash I to Wash II.

7. Wash arrays by submersion and agitation for 2 min in Wash II chamber, then for 2 min in Wash III (transfer the entire slide rack this time).

8. Spin dry by centrifugation in a slide rack in a Beckman GS-6 tabletop centrifuge at 600 RPM for 2 min

9. Scan arrays immediately.

FIG. 16 shows the degree of hybridization achieved via hybridization under constant and homogeneous temperature reaction conditions (squares) versus use of the invented method using a bi-thermal reaction chamber cycled alternately between hybridize and wash on each of the array surfaces. Note that the bi-thermal hybridization reaction achieves completion in under 1 hour while iso-thermal hybridization requires ˜12-16 hours. Also note that bi-thermal hybridization results in nearly 95% hybridization while isothermal hybridization achieves only 35% hybridization.

FIG. 16 shows a dramatic improvement in microarray hybridization provided by the invention. The invention shows a close to 3 fold improvement in total hybridization yield, and achieves this improved yield in 1 hour as opposed to 12-18 hours required by conventional hybridization procedures.

FIG. 17 shows the distribution of probe between hybridization to the desired target spot versus non-specific hybridization/sticking to the array surface (background) versus probe remaining in solution at the end of an 18-hour reaction. Note that the bi-thermal reaction yields nearly 95% hybridization of probe to the desired target spot with less than 3% stuck to the rest of the slide as background and ˜2%. In comparison, note that isothermal reaction yields about 35% hybridization of probe to the desired target spot with less than 25% stuck to the rest of the slide as background and 40% left in solution.

FIG. 17 shows the distribution of labeled Cy3 and Cy5 probe after hybridization is improved by the invention. Specifically, the invention rescues Cy3 and Cy5 probe that would otherwise be sequestered into non-productive fates (such as interactions within the probe mix or sticking to the microarray at an inappropriate location creating background/noise.) FIG. 17 shows that the invention reduces some of the most troubling problems facing microarray hybridization—allowing for greater reliability and sensitivity in processing microarrays.

One of ordinary skill in the art would recognize many other variations, modifications, and alternatives. The above example is merely an illustration, which should not unduly limit the scope of the claims herein. One of ordinary skill in the art would recognize many other variations, modifications, and alternatives. It is also understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A system for facilitating molecular association, the system comprising:
a first substrate comprising a surface, the surface including an array of spots, each of the spots comprising biological material coupled to the substrate;
a second substrate coupled to the first substrate, the second substrate being separated from the first substrate by a predetermined distance;
a fluid disposed between the first substrate and the second substrate and occupying the predetermined distance;
a first energy source coupled to the first substrate to provide a first desired temperature to the first substrate; and
a second energy source coupled to the second substrate to provide a second desired temperature to the second substrate.

2. The system of claim 1 wherein the first substrate is a glass plate.

3. The system of claim 1 wherein the fluid is a liquid.

4. The system of claim 1 wherein the fluid comprises a temperature gradient associated with the first desired temperature and the second desired temperature.

5. The system of claim 1 wherein the first energy source is adjustable to provide an adjustable first desired temperature, the first desired temperature ranging from about 4 degrees Celsius to about 120 degrees Celsius.

6. The system of claim 5 wherein the second energy source is adjustable to provide an adjustable second desired temperature, the second desired temperature ranging from about 4 degrees Celsius to about 120 degrees Celsius.

7. The system of claim 1 wherein the first temperature and the second temperature are adjusted to perform one of a plurlaity of processing steps including hybridization, catalytic or enzymatic processing, ligand binding and/or processing, and antibody binding.

8. The system of claim 1 wherein the first energy source is a Peltier apparatus.

9. The system of claim 1 wherein the first energy source is a heat exchanger apparatus including an incoming fluid line and an outgoing fluid line.

10. The system of claim 1 further comprising a first heat sink coupled between the first substrate and the first energy source and a second heat sink coupled between the second substrate and the second energy source.

11. The system of claim 10 further comprising a thermo couple coupled to the first substrate, the thermo couple being adapted to detect a temperature of the first substrate.

12. The system of claim 10 further comprising a controller coupled to the first energy source and the second energy source.

13. The system of claim 10 further comprising a plurality of fins coupled to the first energy source and a plurality of fins coupled to the second energy source.

14. The system of claim 13 further comprising a first convective source coupled to the plurality of fins coupled to the first energy source and second convective source coupled to the plurality of fins coupled to the second energy source.

15. The system of claim 1 further comprising a seal member disposed between the first substrate and the second substrate to enclose the fluid.

16. The system of claim 1 wherein the first substrate and the second substrate are arranged in a vertical manner to allow the fluid to include a depth parallel to gravity.

17. A method for processing biological materials for diagnostics using molecular association, the method comprising:
disposing fluid between a first substrate and a second substrate, the first substrate being coupled to the second substrate, the first substrate comprising a surface, the surface including an array of spots, each of the spots comprising biological material coupled to the substrate, the second substrate being separated from the first substrate by a predetermined distance, whereupon the fluid occupies the predetermined distance and overlies each of the spots in the array;
applying a first energy to the first substrate to cause a first desired temperature to the first substrate and applying a second energy to the second substrate to cause a second desired temperature to the second substrate; and
causing a temperature gradient between the first substrate and the second substrate through the fluid occupied within the predetermined distance, whereupon the first temperature enhancing a first reaction process at the array of spots and the second temperature enhancing a second reaction process at the second substrate.

18. The method of claim 16 wherein the first temperature is coupled to the second temperature through the temperature gradient.

19. The method of claim 16 wherein the first reaction process is a hybridization process.

20. The method of claim 19 wherein the second reaction process is a stripping process.

21. The method of claim 16 wherein the temperature gradient causes a mixing process of the fluid.

22. The method of claim 16 wherein the temperature gradient is greater than about ten Degrees Celsius.

23. The method of claim 16 wherein the fluid is a pH buffered saline solution which may contain detergents and polymers to promote hybridization and reduce non-specific binding.

24. The method of claim 16 wherein the first substrate and the second substrate are made of glass material.

25. The method of claim 16 wherein the applying the first energy is provided by a Peltier apparatus

26. The method of claim 17 further comprising applying a convective force to the first substate using at least a heat sink.

27. A method for processing biological materials for diagnostics using molecular association and constant mixing, the method comprising:
maintaining fluid between a first substrate and a second substrate, the first substrate being coupled to the second substrate, the first substrate comprising a surface, the surface including an array of spots, each of the spots comprising biological material coupled to the substrate, the second substrate being separated from the first substrate by a predetermined distance, whereupon the fluid occupies the predetermined distance and overlies each of the spots in the array;
causing a temperature gradient between the first substrate and the second substrate through the fluid occupied within the predetermined distance to cause the fluid to circulate between the predetermined distance, whereupon the temperature gradient causing the circulation to mix the fluid within the predetermined distance to maintain a reaction at each of the spots in the array.

28. The method of claim 26 further comprising maintaining the first substrate at a predetermined temperature.

29. The method of claim 26 wherein the fluid is provided in a region defined by a first surface of the first substrate, a second surface of the second substrate, and the predetermined distance between the first surface and the second surface.

30. The method of claim 26 wherein the temperature gradient between the first substrate and the second substrate is at least five degrees Celsius.

31. The method of claim 26 wherein each of the spots is a fragment of nucleic acid or nucleic acid analog.

32. The method of claim 26 further comprising maintaining the first substrate at a predetermined temperature.

33. The method of claim 32 wherein the predetermined temperature is within a range of about 10 degrees centigrade variance.

34. The method of claim 33 wherein the circulation is caused by a convective force within the fluid caused by the temperature gradient.

35. A system for processing biological materials for diagnostics using molecular association and constant mixing, the system including one or more computer codes, the one or more computer codes having:
one or more codes directed to maintaining fluid between a first substrate and a second substrate, the first substrate being coupled to the second substrate, the first substrate comprising a surface, the surface including an array of spots, each of the spots comprising biological material coupled to the substrate, the second substrate being separated from the first substrate by a predetermined distance, whereupon the fluid occupies the predetermined distance and overlies each of the spots in the array; and
one or more codes directed to causing a temperature gradient between the first substrate and the second substrate through the fluid occupied within the predetermined distance to cause the fluid to circulate between the predetermined distance, whereupon the temperature gradient causing the circulation to mix the fluid within the predetermined distance to maintain a reaction at each of the spots in the array.

36. The system of claim 35 comprising one or more codes directed to monitoring a temperature of the fluid.

37. The system of claim 35 comprising one or more codes directed to adjusting a temperature of at least one of the substrates.

38. The system of claim 35 wherein the one or more codes are provided within the memory, the memory being provided on a computer apparatus.

39. The system of claim 38 further comprising an interface coupled between the first substrate and the computer and the second substrate and the computer.

40. The system of claim 35 wherein the temperature gradient is at least ten Degrees Celsius.

41. A method for processing biological materials for diagnostics using molecular association, the method comprising:
disposing fluid between a first substrate and a second substrate, the first substrate being coupled to the second substrate, the first substrate comprising a surface, the surface including an array of spots, each of the spots comprising biological material coupled to the substrate, the second substrate being separated from the first substrate by a predetermined distance, whereupon the fluid occupies the predetermined distance and overlies each of the spots in the array;
applying a first energy to the first substrate to cause a first desired temperature to the first substrate and applying a second energy to the second substrate to cause a second desired temperature to the second substrate;
causing a temperature gradient between the first substrate and the second substrate through the fluid occupied within the predetermined distance;
causing movement within the fluid from the temperature gradient between the first substrate and the second substrate; whereupon the movement within the fluid enhancing a first reaction process at the array of spots.

42. The method of claim 41 wherein the first temperature is coupled to the second temperature through the temperature gradient.

43. The method of claim 41 wherein the first reaction process is a hybridization process.

44. The method of claim 41 wherein the first reaction process is a stripping process.

45. The method of claim 41 wherein the movement is a mixing process of the fluid.

46. The method of claim 41 wherein the temperature gradient is greater than about ten Degrees Celsius.

47. The method of claim 41 wherein the fluid is liquid.

48. The method of claim 41 wherein the first substrate and the second substrate are made of glass material.

49. The method of claim 41 wherein the applying the first energy is provided by a Peltier apparatus

50. The method of claim 41 further comprising applying a convective force to the first substrate using at least a heat sink.

51. A system for processing reactants by manipulating a local concentration of reactants within a reaction chamber in a volume independent manner, the system comprising:
an external force applied to one or more reactants within a fluid in a chamber to move the one or more reactants into a desired reaction zones;
a volume of the fluid maintained independent of an influence of the external force.

52. The system of claim 51 wherein the external force on one or more of the reactants is based on an electronic charge, whereupon the one or more reactants is repelled by a like charge and attracted to an opposite charge.

53. The system of claim 51 wherein the external force is mass based, the mass based includes a force selected from gravity or centrifugation, the force being provided to move the one or more reactants to the desired zone.

54. The system of claim 51 wherein the external force is magnetically based, the magnetically based force includes one or more magnetic fields that drive the one or more reactants to the desired zone.
